(19) United States
(12) Patent Application Publication
(10) Pub. No.: US 2026/0199291 A1
(43) Pub. Date: Jul. 16, 2026

(54) COMPOSITIONS COMPRISING L-ERGOTHIONEINE AND CHRYSOPHANOL FOR REDUCING COPPER-INDUCED CELL DEATH

(71) Applicant: Nutramax Laboratories, Inc., Lancaster, SC (US)

(72) Inventors: David Griffin, Forest Hill, MD (US); Vijaya Varma Indukuri, Indian Land, SC (US)

(73) Assignee: NUTRAMAX LABORATORIES, INC., Lancaster, SC (US)

(21) Appl. No.: 19/443,174

(22) Filed: Jan. 8, 2026

Related U.S. Application Data
(60) Provisional application No. 63/743,839, filed on Jan. 10, 2025.

(51) Int. Cl. A61K 31/4172; A61K 31/122; A61P 1/16

(52) U.S. Cl. CPC A61K 31/4172; A61K 31/122; A61P 1/16

(57) ABSTRACT

A composition for reducing copper-induced liver cell death in a subject, such as a human, dog, or cat, is provided. The composition includes L-ergothioneine and chrysophanol in an amount which provides a synergistic effect in the reduction of copper-induced liver cell death. A ratio of the L-ergothioneine to chrysophanol ranges from 1:1 to 1:8. The composition can include the L-ergothioneine in an amount of 100 μM and the chrysophanol in an amount of 100 μM, for example. Alternatively, the composition can include the L-ergothioneine in an amount of 12.5 μM and the chrysophanol in an amount of 100 μM, for example. The composition can be provided in the form of a liquid, tablet, capsule, soft chew, or another form.

### CROSS REFERENCE TO RELATED APPLICATIONS

This U.S. Non-Provisional patent application claims the benefit of and priority to U.S. Provisional Patent Application Ser. No. 63/743,839 filed Jan. 10, 2025, the contents of which is incorporated herein by reference in its entirety.

### TECHNICAL FIELD

The present invention relates to a composition including L-ergothioneine and chrysophanol, a method for manufacturing a composition including L-ergothioneine and chrysophanol, and a method for ameliorating copper-induced cell death by administering a composition including L-ergothioneine and chrysophanol to a subject.

### BACKGROUND

Copper is an essential mineral that mammals, including, but not limited to, humans, dogs, and cats, need to stay healthy. It has a catalytic impact on wide range of biological processes and is needed to carry out many important functions. For example, mammals use copper for brain development, to make energy, and activate genes. Connective tissues and blood vessels need copper to function properly. Copper also helps support the nervous system and immune system.

When copper ions are absorbed by the small intestine of a mammal, they are transported through the blood and to the liver, since the liver is one organ with an active copper metabolism. However, excess copper in the liver can cause death of liver cells, which leads to adverse heath effects, such as cancers, cardiovascular diseases, and neurodegenerative diseases. Thus, compositions and methods for ameliorating copper-induced liver cell death in a subject are desired.

### SUMMARY

One aspect of the disclosure provides a composition including L-ergothioneine and chrysophanol, preferably in amounts which provide a synergistic effect with regard to reducing copper-induced liver cell death in a subject.

Another aspect of the disclosure provides a method of manufacturing a composition including L-ergothioneine and chrysophanol.

Yet another aspect of the disclosure provides a method for reducing copper-induced cell death by administering a composition including L-ergothioneine and chrysophanol to a subject.

### BRIEF DESCRIPTION OF THE FIGURES

FIG. 1 includes a Table listing inventive and control compositions that were prepared for testing according to a Resazurin Cell Viability assay.

FIG. 2 includes a treatment chart for the Resazurin Cell Viability assay.

FIGS. 3A-3E include graphs of results of the Resazurin Cell Viability assay.

### DETAILED DESCRIPTION OF EXAMPLE EMBODIMENTS

The present invention relates to a composition capable of ameliorating or reducing copper-induced cell death by administering the composition to a subject. The composition includes L-ergothioneine and chrysophanol. The composition preferably includes an amount of the L-ergothioneine and an amount of the chrysophanol which together provide a synergistic effect with regard to ameliorating or reducing copper-induced liver cell death in a subject. The synergistic effect means that the combination of L-ergothioneine and chrysophanol provides a reduction in copper-induced liver cell death, which is greater than an additive effect on reduction in copper-induced liver cell death achieved by the L-ergothioneine alone and the chrysophanol alone. This synergistic effect provides a significant and practical advantage which are not expected in view of the references cited.

According to example embodiments, the composition includes a ratio of the L-ergothioneine to chrysophanol ranging from 1:1 to 1:8. For example, the ratio of the L-ergothioneine to chrysophanol can range from 1:1 to 1:7, 1:1 to 1:6, 1:1 to 1:5, 1:1 to 1:4, 1:1 to 1:3, 1:1 to 1:2, or 1:1 to 1:1. According to an example embodiment, the composition includes the L-ergothioneine in an amount of 100 μM and the chrysophanol in an amount of 100 μM. According to another example embodiment, the composition includes the L-ergothioneine in an amount of 12.5 μM and the chrysophanol in an amount of 100 μM. According to other example embodiments, the composition includes the L-ergothioneine in an amount ranging from 12.5 μM to 100 μM, or 12.5 μM to 50 μM, or 12.5 μM to 25 μM, or 50 μM to 100 μM, or 75 μM to 100 μM.

Another aspect of the disclosure provides a method of manufacturing the composition by combining the L-ergothioneine and the chrysophanol together. For example, the method can include mixing the L-ergothioneine and the chrysophanol according to the ratios or amounts disclosed above.

Yet another aspect of the disclosure provides a method for reducing copper-induced cell death by administering the composition including the L-ergothioneine and chrysophanol, described above, to a subject. The composition can be administered to the subject in various different forms. For example the composition can be administered orally in the form of a liquid, soft chew, tablet, or capsule. Alternatively, the composition can be administered intravenously. The subject can be any animal, including mammals and birds. Mammals include, but are not limited to, humans, dogs, cats, horses, cows, camels, elephants, lions, tigers, bears, seals, and rabbits. In preferred embodiments, the subjects comprise humans, cats, and dogs. Once the composition is administered to the subject, the combination of the L-ergothioneine and chrysophanol can ameliorate copper-induced cell death, such as liver cell death, in the subject.

### Experiment

An experiment was conducted to investigate the ability of a combination of compounds to ameliorate copper-induced cell death, and the potential synergistic effects of the compound combinations, specifically in human liver cells (Hep G2). The compounds tested included L-ergothioneine, chrysophanol, and the combination thereof.

The experiment included obtaining hepatocytes (Hep G2, purchased from ATCC) and co-treating the hepatocytes for 24 hours under standard culture conditions with various doses of chrysophanol and L-ergothioneine. All of the doses were treated along with copper sulfate and some were treated without copper sulfate.

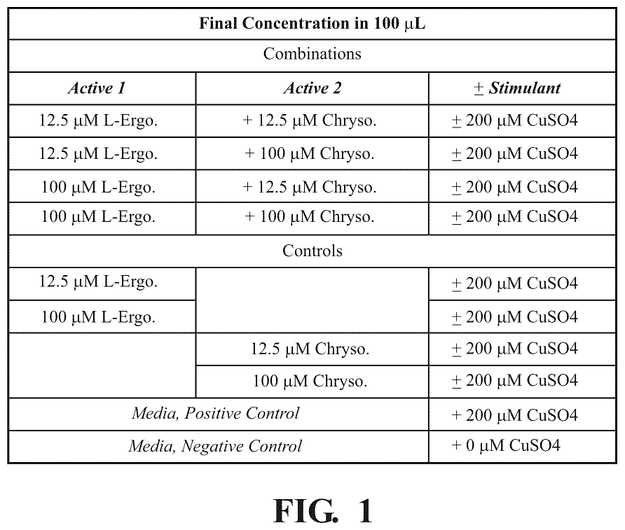

For the experiment, the chrysophanol, also referred to as Chrysophanic Acid, was obtained from SelleckChem, specifically CAT #: S2406, Lot: S240605, Purity: 99.51%. The L-ergothioneine was L-(+)-ergothioneine obtained from Sigma-Aldrich, specifically CAT #: E7521, Lot: BCCD3400, Purity: 99.3%. The copper sulfate was copper (II) sulfate (CuSO₄) obtained from Sigma-Aldrich, specifically CAT #: C1297, Lot: MKCV3136, Purity: 99.0%. FIG. 1 includes a Table listing the inventive and control compositions that were prepared for testing.

At the conclusion of the 24 treatment, a Resazurin Cell Viability assay was performed. The Resazurin Cell Viability test was obtained from Cell Signaling Technology, specifically CAT #: 11884, Lot: 7. The Resazurin Cell Viability assay was performed according to the following manufacturers protocol:

“The Resazurin Cell Viability Kit is a fluorescent assay that detects cellular metabolic activity. The blue nonfluorescent resazurin reagent is reduced to highly fluorescent resorufin by dehydrogenase enzymes in metabolically active cells. This conversion only occurs in viable cells and thus, the amount of resorufin produced is proportional to the number of viable cells in the sample. The resorufin formed in the assay can be quantified by measuring the relative fluorescence units (RFU) using a fluorometer (Ex=530-570 nm, Em=590-620 nm).”

The Resazurin Cell Viability assay described above was conducted over a period of four days. On the first day, Hep G2 cells were seeded in a clear-bottom 96-well culture plate in complete MEM media, with 20,000 Hep G2 cells per well. Three of the wells of the culture plate were left without cells to allow for a ‘Media Blank’ for analysis. The culture plate was incubated overnight to allow for cell attachment.

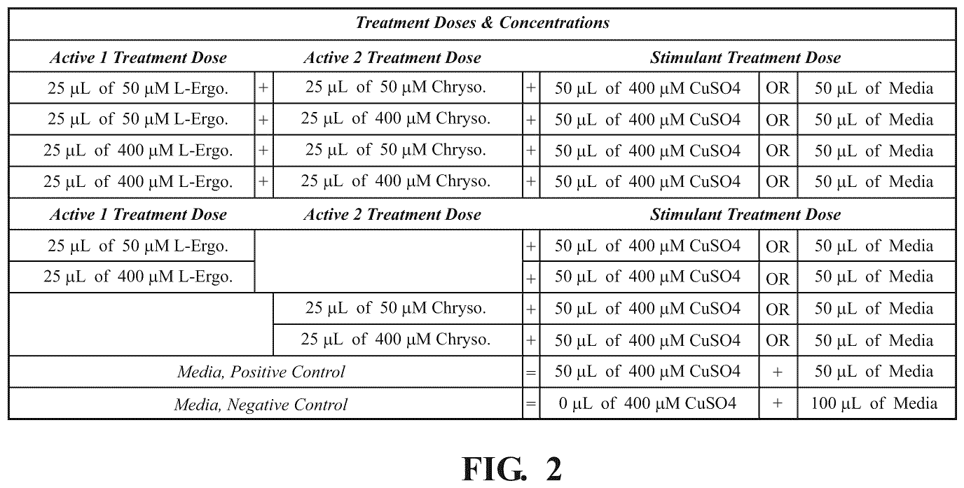

On the second day, the cells were treated according to a treatment chart shown in FIG. 2 (in biological replicates). The test included removing 200 μL media from each well and replacing the removed media with 100 μL of treatment media, as per the treatment plan. The cells were then incubated for 24 hours at standard culture conditions (37° C., 5% CO₂).

On the third day, at 3:16 μm, after 24 hours of incubation, 10 μL of Resazurin solution was added and mixed carefully into each well, wrapped in plate foil to protect from light and incubated for 1 hour. At 4:16 μm, fluorescence of samples were read using a SpectraMax plate reader, protected from light, with an excitation wavelength of 570 nm and emission wavelength of 590 nm.

On the fourth day, the results/data obtained during the test were analyzed using the viability equation presented in a 2016 paper by Santos, et. al. https://doi.org/10.1016/j.cbpc.2016.03.010 (shown below).

$$\text{?} \frac{{RFU}_{SAMPLE} - {RFU}_{\text{?}}}{{RFU}_{\text{?}} - {RFU}_{\text{?}}} \text{?} 100 = \% Viability$$

$$\text{?} \text{indicates text missing or illegible when filed}$$

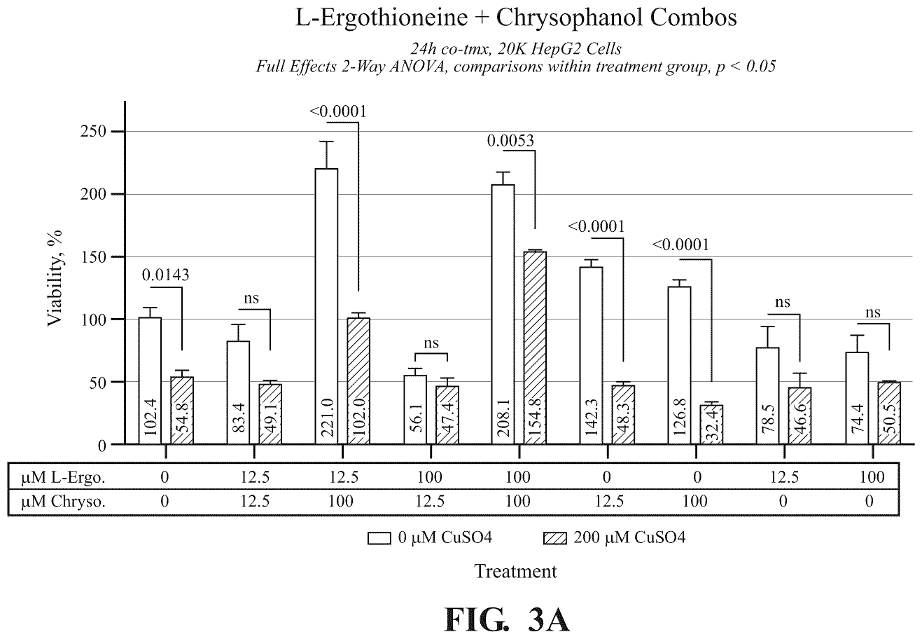

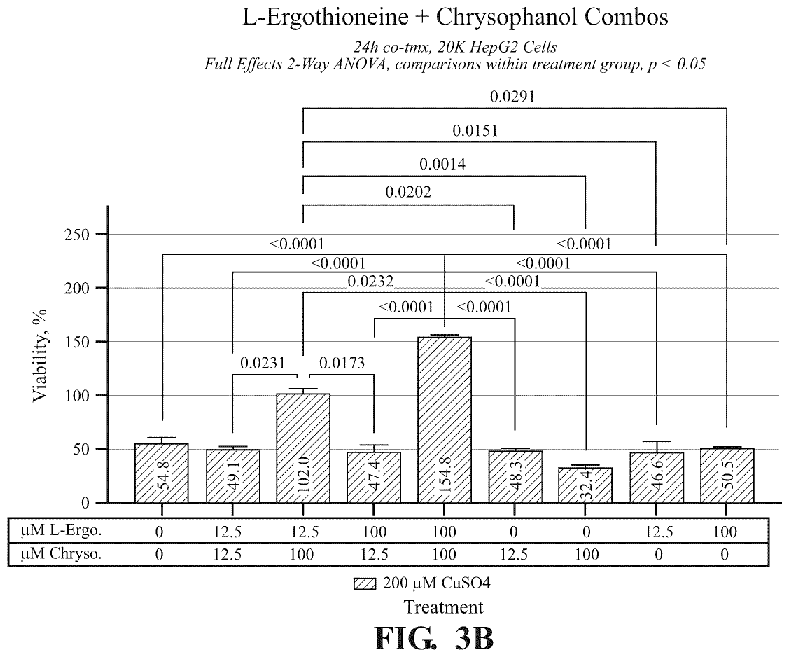

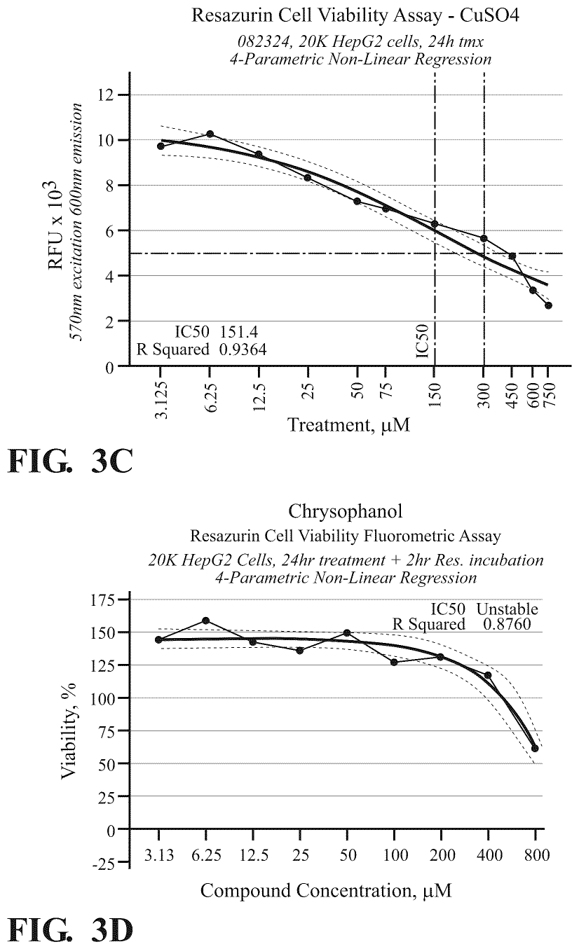

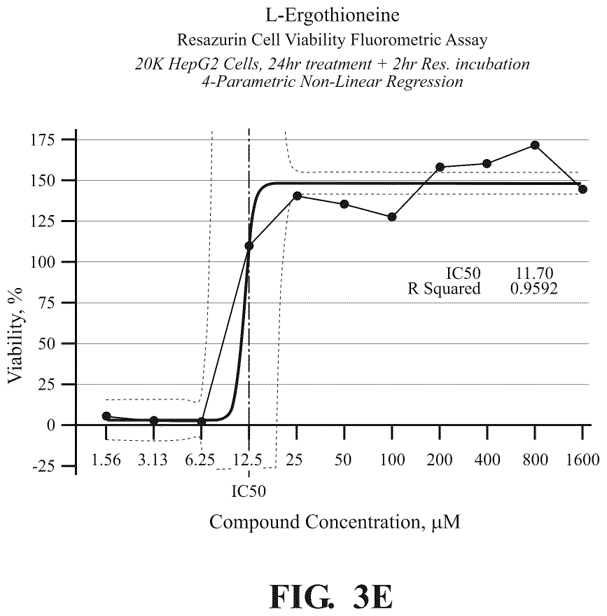

The results, specifically the percent viability of each treatment, were subsequently graphed in GraphPad Prism. FIGS. 3A-3E include the graphs of the test results. Relevant statistical analyses are listed in the graph titles.

The results of the Resazurin Cell Viability assay indicate that a combination of chrysophanol and L-ergothioneine ameliorates the effects of copper-induced liver cell death and exhibits more than an additive synergistic effect compared to each compound alone. The following equations illustrate the presence of the synergistic effect achieved by the combination of chrysophanol and L-ergothioneine.

$$100 μM L - Ergo + Cu = 50.5 \% Viability + 100 μM Chryso . + Cu = 32.4 \% Viability \to Additive effect = 82.9 \%$$

$$100 μM L - Ergo + 100 μM Chryso . + Cu = 54.8 \% Viability \to 154.8 / 82.9 = 1.9 times more viable than additive effects of individual compounds .$$

$$-- -$$

$$12.5 μM L - Ergo + Cu = 48.3 \% Viability + 100 μM Chryso . + Cu = 32.4 \% Viability \to Additive effect = 80.7 \%$$

$$12.5 μM L - Ergo + 100 μM Chryso . + Cu = 102. \% viability \to 102. / 80.7 = 1.3 times more viable than additive effects of individual \\ \\ compounds .$$

Obviously, many modifications and variations of the present invention are possible in light of the above teachings and may be practiced otherwise than as specifically described while within the scope of the following claims.

1. A composition, comprising: L-ergothioneine and chrysophanol.

2. The composition of claim 1 including an amount of L-ergothioneine and chrysophanol which provides a synergistic effect in reduction of copper-induced liver cell death in a subject.

3. The composition of claim 2, wherein the subject is a human, dog, or cat.

4. The composition of claim 1, wherein a ratio of the L-ergothioneine to chrysophanol ranges from 1:1 to 1:8.

5. The composition of claim 4, wherein the L-ergothioneine is in an amount ranging from 12.5 μM to 100 μM.

6. The composition of claim 1 including the L-ergothioneine in an amount of 100 μM and the chrysophanol in an amount of 100 μM.

7. The composition of claim 1 including the L-ergothioneine in an amount of 12.5 μM and the chrysophanol in an amount of 100 μM.

8. A method of manufacturing a composition, comprising the step of; combining L-ergothioneine and chrysophanol.

9. The method of claim 8, wherein the amount of L-ergothioneine and chrysophanol combined provides a synergistic effect in reduction of copper-induced liver cell death in a subject.

10. The method of claim 9, wherein the subject is a human, dog, or cat.

11. The method of claim 8, wherein a ratio of the L-ergothioneine to chrysophanol ranges from 1:1 to 1:8.

12. The method of claim 11, wherein the L-ergothioneine is in an amount ranging from 12.5 μM to 100 μM.

13. The method of claim 8 including the L-ergothioneine in an amount of 100 μM and the chrysophanol in an amount of 100 μM, or including the L-ergothioneine in an amount of 12.5 μM and the chrysophanol in an amount of 100 μM.

14. A method for reducing copper-induced cell death, comprising the step of: administering a composition including L-ergothioneine and chrysophanol to a subject.

15. The method of claim 14, wherein the composition includes an amount of L-ergothioneine and chrysophanol which provides a synergistic effect in reduction of copper-induced liver cell death in the subject.

16. The method of claim 15, wherein the subject is a human, dog, or cat.

17. The method of claim 14, wherein a ratio of the L-ergothioneine to chrysophanol ranges from 1:1 to 1:8.

18. The method of claim 17, wherein the L-ergothioneine is in an amount ranging from 12.5 μM to 100 μM.

19. The method of claim 14 including the L-ergothioneine in an amount of 100 μM and the chrysophanol in an amount of 100 μM, or including the L-ergothioneine in an amount of 12.5 μM and the chrysophanol in an amount of 100 μM.

20. The method of claim 14, wherein the composition is administered in the form of a liquid.
